(19) United States
(12) Patent Application Publication
(10) Pub. No.: US 2009/0143775 A1
(43) Pub. Date: Jun. 4, 2009

(54) MEDICAL LASER HAVING CONTROLLED-TEMPERATURE AND STERILIZED FLUID OUTPUT

(76) Inventors: Ioana M. Rizoiu, San Clemente, CA (US); Dmitri Boutoussov, Dana Point, CA (US)

Correspondence Address: Kenton R. Mullins;Stout, Uxa, Buyan & Mulllins, LLP, 4 Venture, Suite 300, Irvine, CA, 92618, US

(21) Appl. No.: 12/368,276

(22) Filed: Feb. 9, 2009

Related U.S. Application Data
(63) Continuation-in-part of application No. 11/192,329, filed on Jul. 27, 2005.
Application No. 11/192,329 is a continuation-in-part of application No. 11/330,388, filed on Jan. 10, 2006.
Application No. 11/330,388 is a continuation-in-part of application No. 11/033,044, filed on Jan. 10, 2005.
Application No. 11/330,388 is a continuation-in-part of application No. 10/435,325, filed on May 9, 2003, now Pat. No. 7,320,594.
Application No. 10/435,325 is a division of application No. 09/997,550, filed on Nov. 27, 2001, now Pat. No. 6,561,803.
Application No. 09/997,550 is a continuation of application No. 09/256,697, filed on Feb. 24, 1999, now Pat. No. 6,350,123.
Application No. 09/256,697 is a continuation-in-part of application No. 08/985,513, filed on Dec. 5, 1997, now abandoned.
Application No. 08/985,513 is a continuation of application No. 08/522,503, filed on Aug. 31, 1995, now Pat. No. 5,741,247.
(60) Provisional application No. 60/591,934, filed on Jul. 27, 2004.
(60) Provisional application No. 60/645,427, filed on Jan. 19, 2005.
(60) Provisional application No. 60/696,475, filed on Jul. 1, 2005.
(60) Provisional application No. 60/709,714, filed on Aug. 19, 2005.
(60) Provisional application No. 60/535,110, filed on Jan. 8, 2004.

(51) Int. Cl. A61B 18/20; A61B 18/04; A61B 18/02

(52) U.S. Cl. 606/13; 607/104

(57) ABSTRACT

A method and apparatus are described for controlling temperature of a fluid used with electromagnetic energy in medical and dental laser procedures. Received fluid is passed through a heat exchanger, and heat is added to or removed from the fluid depending upon a desired effect, which may be influenced by a temperature setting. Temperature of output fluid is sensed, and heating or cooling is controlled in order to maintain output fluid temperature in a desired range. Ultraviolet radiation is used to sterilize the fluid.

### CROSS-REFERENCE TO RELATED APPLICATIONS

This application is a continuation-in-part of U.S. application Ser. No. 11/192,329 (Att. Docket BI9803P), filed Jul. 27, 2005 and entitled MEDICAL LASER HAVING CONTROLLED-TEMPERATURE AND STERILIZED FLUID OUTPUT, the entire contents all of which are expressly incorporated herein by reference. U.S. application Ser. No. 11/192,329 claims the benefit of U.S. Provisional Application No. 60/591,934 (Att. Docket BI9803PR), filed Jul. 27, 2004 and entitled MEDICAL LASER HAVING DUAL-TEMPERATURE FLUID OUTPUT, the entire contents of which are incorporated herein by reference. This application is also a continuation-in-part of U.S. application Ser. No. 11/330,388 (Att. Docket BI9914P), filed Jan. 10, 2006 and entitled FLUID CONDITIONING SYSTEM, the entire contents all of which are expressly incorporated herein by reference. U.S. application Ser. No. 11/330,388 claims the benefit of U.S. Provisional Application No. 60/645,427 (Att. Docket BI9694CIPPR), filed Jan. 19, 2005 and entitled FLUID CONDITIONING SYSTEM, U.S. Provisional Application No. 60/696,475 (Att. Docket BI9903PR), filed Jul. 1, 2005 and entitled FLUID CONDITIONING SYSTEM, and U.S. Provisional Application No. 60/709,714 (Att. Docket BI9914PR), filed Aug. 19, 2005 and entitled FLUID CONDITIONING SYSTEM, the entire contents all of which are expressly incorporated herein by reference. U.S. application Ser. No. 11/330,388 is a continuation-in-part of U.S. application Ser. No. 11/033,044 (Att. Docket BI9694P), filed Jan. 10, 2005 and entitled FLUID CONDITIONING SYSTEM, the entire contents of which are expressly incorporated herein by reference. U.S. application Ser. No. 11/033,044 claims the benefit of the above-referenced U.S. Provisional Application No. 60/535,110. U.S. application Ser. No. 11/330,388 is also a continuation-in-part of U.S. application Ser. No. 10/435,325 (Att. Docket BI9002CNCPCNDIV), filed May 9, 2003 and entitled FLUID CONDITIONING SYSTEM, which is a divisional of U.S. application Ser. No. 09/997,550 (Att. Docket BI9002CNCPCN), filed Nov. 27, 2001 and entitled FLUID CONDITIONING SYSTEM, issued as U.S. Pat. No. 6,561,803, which is a continuation of U.S. application Ser. No. 09/256,697 (Att. Docket BI9002CONCIP), filed Feb. 24, 1999 and entitled FLUID CONDITIONING SYSTEM, issued as U.S. Pat. No. 6,350,123, which is a continuation-in-part of U.S. application Ser. No. 08/985,513 (Att. Docket BI9001CON), filed Dec. 5, 1997 and entitled FLUID PARTICLES FOR ELECTROMAGNETICALLY INDUCED CUTTING, now abandoned, which is a continuation of U.S. application Ser. No. 08/522,503 (Att. Docket BI9001P), filed Aug. 31, 1995 and entitled ATOMIZED FLUID PARTICLES FOR ELECTROMAGNETICALLY INDUCED CUTTING, issued as U.S. Pat. No. 5,741,247, the entire contents all of which are expressly incorporated herein by reference.

### BACKGROUND OF THE INVENTION

1. Field of the Invention

The present invention relates generally to electromagnetic energy emitting devices and, more particularly, to a medical laser having associated therewith a fluid output.

2. Description of Related Art

Assuring comfort of dental patients can be a paramount consideration in the operation of many dental practices. As perhaps a consequence of this consideration, belt-driven drills were replaced with high-speed air-driven drills for cutting hard dental tissue such as enamel and dentin, to thereby provide for greater efficiency and reliability and further to provide an increased measure of comfort to the dental patient.

More recently, laser devices have been used in dental cutting applications, which devices may further increase efficiency, reliability and dental patient comfort. Laser cutting of hard dental tissue can be almost painless. Certain laser cutting methods may employ a laser dental handpiece that emits a water spray to assist with laser cutting by cooling cut surfaces and removing debris associated with the laser cutting procedures.

For patients with enhanced sensitivity to cold, for example, the room-temperature water spray may be uncomfortable, although the cutting itself may cause relatively little pain. Such patients may potentially have a pre-disposition or preference for warmed water, should such water ever be made available in procedures, such as in the context of, for example, laser-assisted dental procedures.

Other patients may possess a preference in general for chilled water (e.g., cooled drinking water) as opposed to room-temperature water. These patients potentially may have a preference or pre-disposition for cooled water, should this type of reduced-temperature water be made available to the patient in procedures, such as dental laser procedures involving the patient.

In the context of laser assisted soft tissue procedures, such as soft tissue cutting, as well as in laser-assisted operations applicable to non-dental hard tissues, such as cutting of bones, bleeding or hemorrhea may occur. Various methods and apparatuses have been developed in the prior art for controlling, attenuating, or virtually eliminating these effects.

### SUMMARY OF THE INVENTION

The present invention addresses deficiencies in the prior art by providing a method of controlling a temperature of a fluid emitted by an electromagnetic energy emitting device, such as a medical laser. In accordance with an aspect of the present invention, a method comprises selectively providing relatively cold water (i.e., colder than room temperature) and relatively warm water (i.e., warmer than room temperature) for use in combination with an output from a medical laser. The water can be used in the form of a spray that is combined with the output from the medical laser. The relatively cold water may, for example, provide patient comfort or aid in reducing bleeding by causing constriction of blood vessels. Likewise, the relatively warm water may, for example, provide comfort to other patients in other contexts.

The present invention recognizes that heating water may accelerate growth of bacteria in a water line supplying the water, and further recognizes that water used in dental procedures should be clean and, for surgical purposes, sterile. To address scenarios wherein bacterial growth promoted by heating water works at cross-purposes with, for example, sterility requirements, an aspect of the present invention provides a method that can supply sterilized, temperature-controlled water for use in laser dental cutting operations.

An implementation of the invention herein disclosed comprises providing a medical laser capable of receiving a fluid input and producing a fluid output. The implementation further comprises receiving the fluid input, controlling a temperature of the fluid, and producing the fluid output, for example, in combination with an output from the medical laser. Another implementation of the method further comprises sterilizing the fluid.

One embodiment of the present invention comprises a medical laser having a receiving tube capable of receiving a fluid from a fluid input line. The embodiment further comprises a heat exchanger capable of at least one of adding heat to the fluid and removing heat from the fluid. The embodiment still further comprises an output capable of receiving the fluid from the heat exchanger and of expelling the fluid, for example, in combination with an output from the medical laser. Another embodiment of the medical laser comprises a sterilizing device capable of sterilizing the fluid.

While the apparatus and method has or will be described for the sake of grammatical fluidity with functional explanations, it is to be expressly understood that the claims, unless expressly formulated under 35 U.S.C. 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. 112 are to be accorded full statutory equivalents under 35 U.S.C. 112.

Any feature or combination of features described herein are included within the scope of the present invention provided that the features included in any such combination are not mutually inconsistent as will be apparent from the context, this specification, and the knowledge of one skilled in the art. For purposes of summarizing the present invention, certain aspects, advantages and novel features of the present invention are described herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE FIGURES

FIG. 1 is a flow diagram describing an implementation of a method of controlling temperature of a fluid and of sterilizing the fluid in accordance with the present invention;

FIG. 2 is a pictorial diagram of an embodiment of an apparatus that controls fluid temperature and sterilizes the fluid according to the present invention;

FIG. 3 is a block diagram of an embodiment of a medical laser fluid temperature controller illustrated as a part of FIG. 2 and capable of controlling fluid temperature; and

FIG. 4 is a block diagram showing a fluid output used in combination with an electromagnetic energy source in accordance with the present invention.

### DETAILED DESCRIPTION OF THE INVENTION

Reference will now be made in detail to embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. It is to be understood and appreciated that the process steps and structures described herein do not cover a complete process flow for operation of electromagnetic energy emitting devices. The present invention may be practiced in conjunction with various techniques that are conventionally used in the art, and only so much of the commonly practiced process steps and structures are included herein as are necessary to provide an understanding of the present invention. The present invention has applicability in the field of electromagnetic energy devices in general. For illustrative purposes, however, the following description pertains to a medical laser device and a method of controlling temperature and sterility of a fluid emitted by the medical laser device in surgical applications.

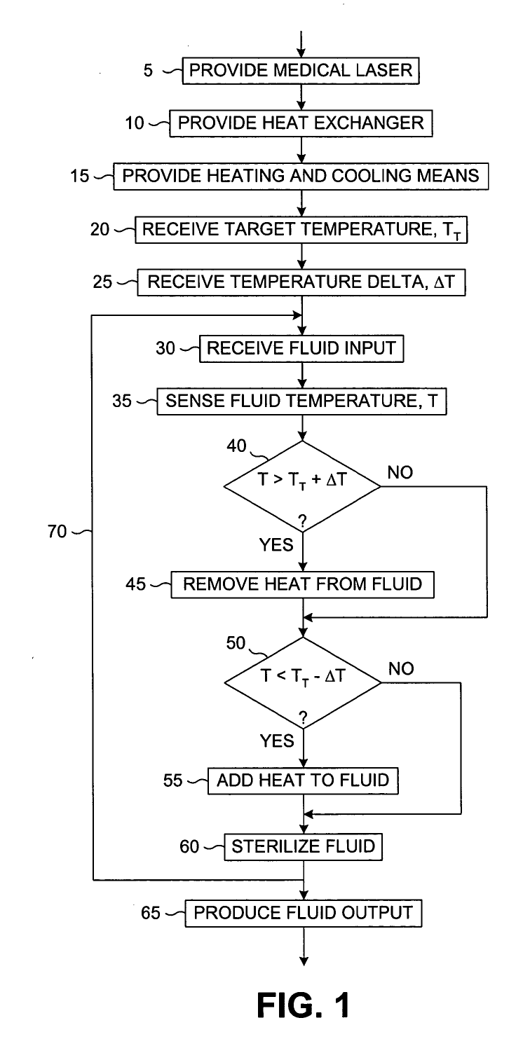

Referring more particularly to the drawings, FIG. 1 is a flow diagram describing an implementation of a method of controlling a fluid temperature and sterilizing the fluid emitted by an electromagnetic energy emitting device, such as a medical laser, according to an embodiment of the present invention. The method can have applicability to a medical laser, which can be provided at step 5 of the method and which may be used in, for example, dental applications. Examples of electromagnetic energy emitting systems (e.g., medical lasers and associated delivery systems) with which methods and structures of the present invention may be combined (e.g., including modification-based combinations as would be recognized by those skilled in the art) are provided in U.S. application Ser. No. 60/696,475, filed Jul. 1, 2005 and entitled FLUID CONDITIONING SYSTEM, the entire contents of which are incorporated herein by reference. An embodiment of an electromagnetic energy emitting device (e.g., medical laser device) such as described in the just-referenced patent application, includes a fluid line from which is received fluid (e.g., water) that in certain instances is mixed with air in a vicinity of a handpiece, for example, to create a spray (e.g., atomized distribution of water particles), wherein the fluid is directed, in combination with emitted electromagnetic energy toward, a target surface. Typically, the fluid comprises a water spray that is applied with laser energy to facilitate treatment or cutting of a target surface, such as a tooth surface.

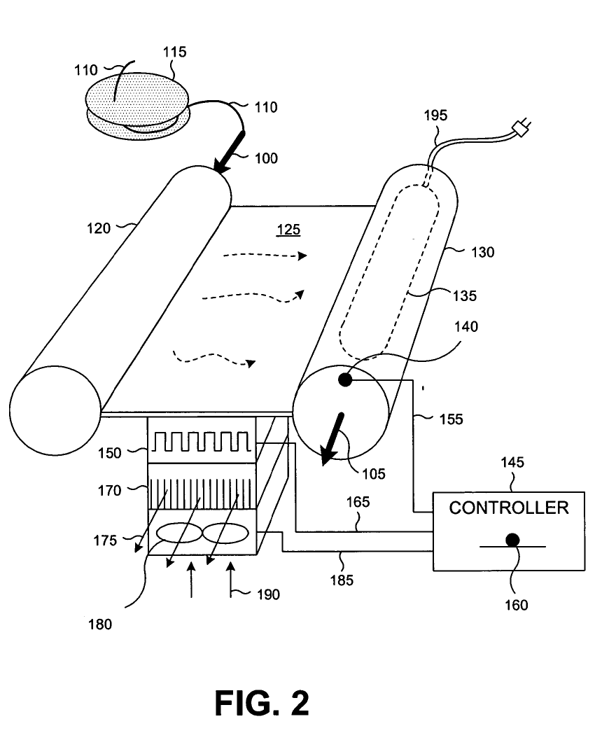

The implementation illustrated in FIG. 1 continues with the provision of a heat exchanger at step 10. The heat exchanger may be chosen, for example, to be relatively thin in order to promote efficient heat transfer, and baffles or the like may be provided within the heat exchanger to encourage turbulent mixing of fluid passing therethrough. A device capable of heating and cooling fluid, which may be implemented by a thermo-electric cooler in one embodiment, further can be provided at step 15 of the implementation. The discussion below with reference to FIG. 2 describes an embodiment that includes a heat exchanger, a heating component and a cooling component suitable for use with implementations of the method described in FIG. 1.

A target temperature T_{T} can be received at step 20, the target temperature T_{T} representing a desired temperature for a fluid (e.g., water) to be emitted by a medical laser according to the current example of the present invention. A tolerance value of temperature, which may be referred to as a temperature delta ΔT, can be received at step 25. At step 30, a fluid can be received from a fluid input or otherwise accessed. In exemplary implementations, part or all of the fluid or fluid components (e.g., fluid conditioning fluid components) may be received from a pressurized water line and/or accessed from a local reservoir (e.g., canister attached to the laser). According to a typical embodiment, the fluid comprises water that may or may not be treated using methods described in the above-referenced U.S. Application No. 60/696,475, filed Jul. 1, 2005 and entitled FLUID CONDITIONING SYSTEM. In typical embodiments, the fluid may be filtered in, for example, a water line and/or a cartridge or other type of filtration system. An exemplary implementation of a suitable filter can comprise, for example, pores no greater than about 5 μm in diameter. The fluid may be received, for example, into an input tube from which the fluid passes through the heat exchanger.

At an output of the heat exchanger the fluid may pass into an output tube where a temperature T of the fluid is measured (i.e., sensed) at step 35. The measured temperature T is compared at step 40 of the implementation with an upper limit that may be chosen, according to an exemplary embodiment, to be a sum of the target temperature T_{T} and the temperature delta ΔT. If the measured temperature T is greater than the upper limit, then heat is removed from the fluid at step 45. The cooling device may interact with the heat exchanger according to principles well understood to those skilled in the art to remove heat from the fluid, thereby tending to lower its temperature. The measured temperature T is further compared at step 50 with a lower limit that may assume a value in a typical embodiment of the target temperature T_{T} minus the temperature delta ΔT. If the measured temperature T is less than the lower limit, then heat may be added to the fluid at step 55 of the implementation.

Exemplary values for the target temperature and the temperature delta ΔT may depend upon a procedure to be performed. Some representative examples follow. For enamel cutting, the target temperature may range from about 27° C. to about 80° C. with a temperature delta ΔT of about 2° C. and with a possibility of adjusting the given temperatures to compensate for different thermal losses which may occur based upon the different types and lengths of fluid lines that may be used. For dentin cutting, target temperatures may range from about 30° C. to about 77° C. with a temperature delta ΔT of about 2° C. and with a possibility of adjusting the given temperatures to compensate for different thermal losses which may occur based upon the different types and lengths of fluid lines that may be used. For soft tissue cutting, target temperatures in a range of about 1° C. to about 15° C., or 25° C. to about 85° C., may be employed with a temperature delta ΔT of about 2° C. and with a possibility of adjusting the given temperatures to compensate for different thermal losses which may occur based upon the different types and lengths of fluid lines that may be used.

The fluid is sterilized at step 60. Simple methods of sterilization, such as methods that employ heating, may not be optimal or applicable in the present instance because such heating would interfere with the intention to maintain a fluid temperature that is comfortable for patients. One technique that may not so interfere can comprise irradiating the fluid with ultraviolet light, thereby disinfecting the fluid. The ultraviolet light may have a wavelength of about 254 nm. The fluid output is produced at step 65. Typically, the fluid output is subsequently employed in a mixture with air to create a spray that may be used to assist laser cutting.

If, at step 40, the measured temperature T is not greater than the upper limit, then the implementation of the method continues at step 50 with comparison of the measured temperature T with the lower limit. If, at step 50, the measured temperature T is not less than the lower limit, then the method continues at step 60 with sterilization of the fluid.

The implementation of the method depicted in FIG. 1 includes a feedback loop 70, which is intended to illustrate that the sensing (i.e., measuring) of fluid temperature (e.g., fluid temperature at the fluid output ) can be a continuous process. One, the other, or both of the heating component and the cooling component may be controlled (e.g., by a controller) to maintain a temperature of the output fluid in a desired range. The limits of the range may be defined, for example, as described above by a target temperature T and a temperature delta ΔT.

According to an alternative implementation of the method of the present invention, limits of a desired temperature range may be defined by a low temperature and a high temperature. For example, the low temperature may play a role similar to that played by the target temperature T minus temperature delta ΔT at step 50 of the implementation described in FIG. 1. Similarly, the high temperature may play a role similar to that of the sum of the target temperature T and the temperature delta ΔT at step 40 of the above-described implementation.

Certain implementations may exclude steps 40 and 45, while other implementations may exclude steps 50 and 55, while still further implementations of the inventive method may exclude step 60. In modified implementations, determinations that the measured temperature T is not greater than the upper limit (i.e., step 40) can result in the method advancing to, for example, step 60, step 30, or step 65. In other modified implementations, determinations that the measured temperature T is not less than the upper limit (i.e., step 50) can result in the method advancing to, for example, step 60, step 30, or step 65.

An implementation of a method of controlling a fluid temperature, in accordance with for example the above-described flow diagram, can include selectively providing relatively cold fluid (e.g., water below room temperature) and relatively warm fluid (e.g., water above room temperature) for use (e.g., as a spray or water spray) in combination with an output from a medical laser.

Cooled matter (e.g., fluid) may, for example, provide patient comfort or may provide aid in reducing bleeding by way of an encouragement of constriction of blood vessels. When warmed, matter (e.g., fluid) may provide, for example, comfort or benefit to other patients in other contexts. In certain embodiments, cooled matter (e.g., air and/or water below room temperature) may be applied to a tissue, for example, to control bleeding, which bleeding may have been caused by cutting, ablating, or other trauma inflicted on the tissue. The cooled matter (e.g., fluid) may be applied, for example, to an eye to slow down or stop bleeding following an ablation procedure, such as a cutting procedure performed with a laser.

The temperature of the fluid and/or duration of application of the fluid to the tissue may be controlled, for example, to bring about a desired effect, such as attenuation of bleeding. In an exemplary implementation, a temperature and duration are selected or controlled to attenuate or eliminate bleeding of a tissue while not causing permanent necrosis of cells of the tissue.

The present invention recognizes that heating water may accelerate growth of bacteria in a water line supplying the water, and further recognizes that water used in dental procedures should be clean and, for surgical purposes, sterile. To address scenarios wherein bacterial growth promoted by heating water works at cross-purposes with, for example, sterility requirements, an aspect of the present invention can provide temperature-controlled water in a sterile state for use in laser medical (e.g., dental) cutting operations (cf. step 60).

An embodiment of the present invention can comprise a medical laser, which may be used for cutting hard dental tissue (e.g., enamel and dentin), soft tissue, or bone. The medical laser may comprise a handpiece that incorporates an apparatus capable of producing a sterile fluid output having a controlled temperature according to the method described in FIG. 1.

FIG. 2 illustrates an exemplary embodiment of such an apparatus, wherein the apparatus can comprise one or more of an input tube 120, a heat exchanger 125, an output tube 130, and an ultraviolet lamp 135. The embodiment further can comprise one or more of a temperature sensor 140, a medical laser fluid temperature controller 145, and a thermo-electric cooler 150. According to an alternative embodiment, the just-listed elements of the apparatus may be provided in a unit upstream from the medical laser handpiece, for example, in tandem with a water line.

In a typical operating mode, the apparatus receives fluid from a fluid input 100. The fluid may be supplied, for example, from a water line 110. Fluid (e.g., water) from the water line 110 may be filtered with a filter 115, which may comprise a 5 μm filter in a typical embodiment. The fluid may be received into the input tube 120 and, subsequently, passed through the heat exchanger 125. The heat exchanger 125 typically is chosen to be relatively thin in order to promote efficient transfer of heat between fluid inside the heat exchanger 125 and the heating and cooling component, which, as embodied in the illustrated example, comprises the thermo-electric cooler 150 disposed externally to the heat exchanger 125.

Temperature-adjusted fluid can pass from the heat exchanger 125 into the output tube 130, which can comprise the ultraviolet lamp 135 disposed therein. Power to the ultraviolet lamp 135 may be provided from a conventional electrical power source 195. Fluid exposed to ultraviolet radiation from the ultraviolet lamp 135 may be sterilized by the radiation. The temperature sensor 140 may be disposed inside the output tube 130 at a position near a point of fluid output 105 where fluid exits the output tube 130. In one embodiment, the fluid then passes to a mixing chamber where it mixes with air according, for example, to known methods of operating medical lasers. In other embodiments, the fluid can be outputted from the laser without mixing, can be mixed with other components, and/or can be mixed (e.g., combined with pressurized air) after being outputted from the laser. The fluid can be outputted toward a target surface (e.g., and combined with electromagnetic energy) in accordance with any of the various methods and structures contained or suggested in any of the documents and related documents referenced herein. In another embodiment, the fluid can pass through a section of a water line to a medical laser handpiece.

The temperature sensor 140 may measure a temperature of the fluid at the point of fluid output 105. An electrical signal according to the temperature measurement may be passed to the medical laser fluid temperature controller 145 by way of a sensing connection 155. The medical laser fluid temperature controller 145, which may comprise a temperature regulator 160, is capable of generating a first control signal that is coupled to the thermoelectric cooler 150 by a first control connection 165. The temperature regulator 160 may receive, for example, a desired temperature from a user input (not shown) and, further, may receive a temperature delta ΔT from the user input. According to another embodiment, the temperature regulator 160 receives a high temperature input and a low temperature input from the user input.

In another embodiment, a microprocessor, which may control or communicate with one or more of the apparatus and the medical laser, executes instruction sequences comprising temperature values (e.g., preset temperature values) for the high and low temperatures or for the desired temperature and the temperature delta ΔT. Execution of these instruction sequences minimally may cause, for example, the microprocessor to pass the preset temperature values to the temperature regulator. According to yet another embodiment, the just-listed temperature inputs can be determined by instruction sequences executed by the microprocessor according to a medical procedure being performed. For example, the instruction sequences may cause the microprocessor to determine a relatively low temperature range when a soft tissue or vascularized bone laser procedure is to be performed. When a tooth-cutting procedure is to be performed, the instruction sequences may cause the microprocessor to determine, for example, a relatively high temperature in order to enhance comfort of a patient undergoing the procedure.

The thermoelectric cooler 150 may have associated therewith a heat sink 170 capable of dissipating heat extracted from the fluid by the thermo-electric cooler 150. A fan 180 may be controlled by a second control connection 185 with the medical laser fluid temperature controller 145. The fan may, according to one example, receive input air 190. The input air 190 may pass over fins of the heat sink 170 where the air may take up heat and leave the heat sink 170 as exit air 175. According to another example, the thermo-electric cooler 150 generates heat, a portion of which is transferred to the fluid by the heat exchanger 125. Excess heat generated in the process may be dissipated in the heat sink 170 and carried off in exit air 175.

The medical laser fluid temperature controller 145 may continuously monitor a measured temperature of the fluid as reported by the temperature sensor 140 and received by way of the sensing connection 155. Accordingly, the medical laser fluid temperature controller 145 may generate signals on first and second control connections 165 and 185 to control the thermo-electric cooler 150 and the fan 180. By so doing, the medical laser fluid temperature controller 145 may cause the measured temperature of the output fluid 105 to change according to a desired temperature range received by the temperature regulator 160.

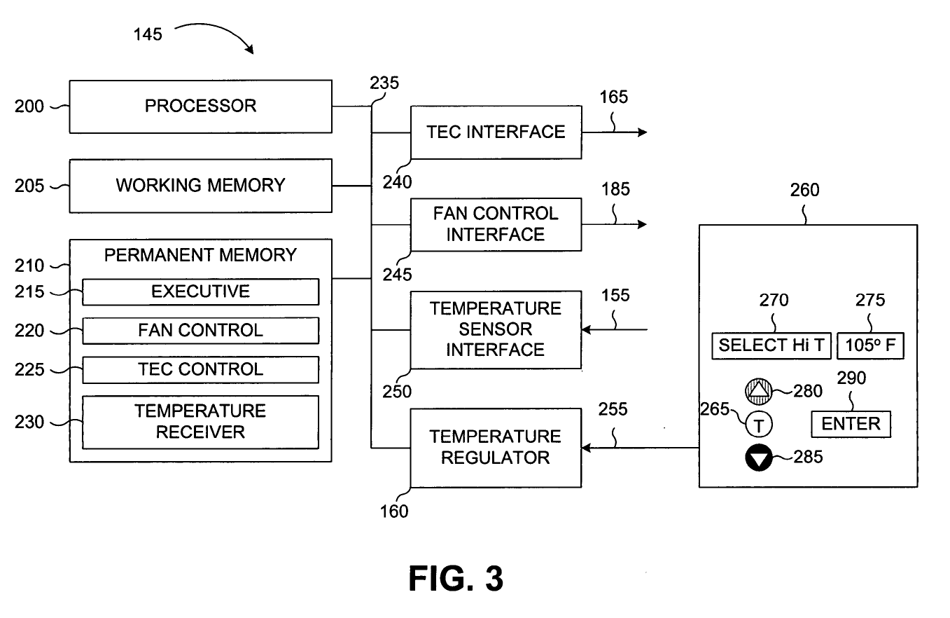

FIG. 3 is a block diagram of an embodiment of a medical laser fluid temperature controller 145 capable of controlling fluid temperature in a medical laser device. The illustrated embodiment comprises a processor 200 (e.g., a microprocessor), working memory 205, permanent memory 210, and a temperature sensor interface 250. The embodiment further comprises a thermo-electric cooler (TEC) interface 240, a fan control interface 245, and a temperature regulator 160. The aforementioned elements of the embodiment are interconnected by a system bus 235 that facilitates communication among the elements. The illustrated embodiment further comprises a user input panel 260. In some embodiments, the processor 200, the working memory 205 (e.g., random access memory (RAM)), the permanent memory 210, and other system elements, such as a clock (not shown), may be implemented on a single microcontroller chip as an application-specific integrated circuit (ASIC). In other embodiments, the thermo-electric control interface 240, the fan control interface 245, the temperature sensor interface 250, and the temperature regulator 160 further may be implemented on the same chip. In yet other embodiments, the latter four elements may be implemented on a companion chip to the microcontroller chip. Each of these and other equivalent implementations are contemplated by the present invention.

The permanent memory 210, which may be programmed at a time of manufacture of an ASIC, for example, may have instruction sequences programmed therein that may, when executed by the processor 200, cause the medical laser fluid temperature controller 145 to perform functions according to the present invention. These instruction sequences in the illustrated embodiment include an executive instruction sequence 215, a fan control instruction sequence 220, a thermo-electric control instruction sequence 225, and a temperature receiver instruction sequence 230. The permanent memory 210 in other embodiments may be programmed with additional instruction sequences related, for example, to laser handpiece functions tangential to or outside the immediate scope of the present invention. For example, the permanent memory 210 may contain an instruction sequence that controls laser pulse width according to a user input as well as similar types of instruction sequences related to operation of medical lasers. The executive instruction sequence 215 minimally may cause the processor 200 to schedule and coordinate the instruction sequences described herein as well as additional instruction sequences not herein described.

According to an exemplary mode of operation, the embodiment of FIG. 3 receives an input from a user on a user input line 255. The user input line may be connected to the user input panel 260 which may have disposed thereon an input temperature selection button 265, a temperature increment button 280 and a temperature decrement button 285. A function display strip 270, such as a liquid crystal display (LCD), may display a function chosen according to the temperature selection button 265, and a numerical temperature value may be displayed in a temperature display strip 275. An ENTER key 290 may generate a user input on the user input line 255 according to the configuration entered on the user input panel 260. For example, a user may repeatedly press the temperature selection button 265 to cause the function display strip 270 to cycle through available function settings. An example might include “SELECT Hi T,” displayed on the function display strip 270 to invite selection of a high temperature setting. Similarly, display of “SELECT Lo T” may invite selection of a low temperature setting. A display of “SEL TGT T” may invite setting of a target temperature value, and so on. Available function settings may be scrolled rather than shortened. For example, “SELECT TARGET TEMPERATURE” may be scrolled on the display rather than displaying the shorter “SEL TGT T.” Once a function is selected, the temperature increment button 280 and temperature decrement key 285 may be used, for example, to cause a desired temperature to be displayed in the temperature display strip 275. When the desired temperature is displayed, the user may press the ENTER key 290 as mentioned to cause the selected setting to be transferred, for example, to the temperature regulator 160 over the user input line 255.

It should be emphasized that the form of the user input panel 260 illustrated in FIG. 3 is only one of many possible examples of user input devices contemplated by the present invention. For example, a conventional 12-button keypad or a touch screen could be used. Indeed, according to one exemplary mode of operation, a user panel for entering temperature data can be combined with, or subsumed by, a preexisting user input panel (not shown) that may be used to enter information regarding other medical laser functions. Fluid temperatures associated with these other medical laser functions in such exemplary modes may be selected automatically by the processor 200 according to instruction sequences stored in permanent memory 210.

When a user input is received on user input line 255, the temperature regulator may communicate information from the user input to the processor 200. For example, the temperature regulator 160 may communicate a high temperature setting and a low temperature setting to the processor 200, the high and low temperature settings defining a desired range of fluid temperature. The processor 200 may store the high and low temperature settings in working memory 205.

The processor 200 may execute the temperature receiver instruction sequence 230 that minimally may cause the processor to receive a measured temperature value from sensing connection 155 (see also description above with reference to FIG. 2) through the temperature sensor interface 250. The temperature receiver instruction sequence 230 further minimally may cause the processor 200 to compare the measured temperature value with, for example, the high temperature setting and the low temperature setting stored in working memory 205. If the measured temperature value is lower than the low temperature setting, then the processor 200 may execute the thermoelectric control instruction sequence 225. The thermoelectric control instruction sequence 225 minimally may cause the processor to control the TEC 150 (FIG. 2) through the TEC interface 240 to add heat to the fluid through the heat exchanger 125. If the measured temperature value is higher than the high temperature setting, then the processor 200 may again execute the thermoelectric control instruction sequence 225. In this instance, the thermoelectric control instruction sequence 225 minimally may cause the processor 200 to control the TEC 150 (FIG. 2) through the TEC interface 240 to remove heat from the fluid through the heat exchanger 125. The processor 200, further, may execute the fan control instruction sequence 220 that minimally may cause the processor 200 to send control signals to operate the fan 180 (FIG. 2) through the fan control interface 245. Operating the fan may cause input air 190 to pass through the heat sink 170, thereby causing the air to remove heat from the TEC before leaving the heat sink 170 as exit air 175.

In certain embodiments, the methods and apparatuses of the above embodiments can be configured and implemented for use, to the extent compatible and/or not mutually exclusive, with existing technologies including any of the above-referenced apparatuses and methods. Corresponding or related structure and methods described in the following patents assigned to BioLase Technology, Inc., are incorporated herein by reference in their entireties, wherein such incorporation includes corresponding or related structure (and modifications thereof) in the following patents which may be (i) operable with, (ii) modified by one skilled in the art to be operable with, and/or (iii) implemented/used with or in combination with any part(s) of, the present invention according to this disclosure, that/those of the patents, and the knowledge and judgment of one skilled in the art: U.S. Pat. No. 5,741,247; U.S. Pat. No. 5,785,521; U.S. Pat. No. 5,968,037; U.S. Pat. No. 6,086,367; U.S. Pat. No. 6,231,567; U.S. Pat. No. 6,254,597, U.S. Pat. No. 6,288,499; U.S. Pat. No. 6,350,123; U.S. Pat. No. 6,389,193; U.S. Pat. No. 6,544,256; U.S. Pat. No. 6,561,803; U.S. Pat. No. 6,567,582; U.S. Pat. No. 6,610,053; U.S. Pat. No. 6,616,447; U.S. Pat. No. 6,616,451; U.S. Pat. No. 6,669,685; and U.S. Pat. No. 6,744,790 all of which are commonly assigned and the entire contents of which are incorporated herein by reference.

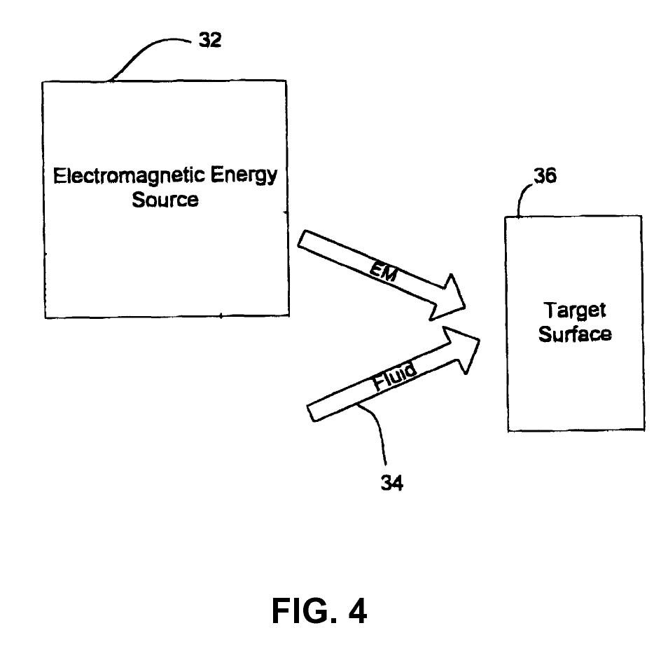

For example, one implementation may be useful for optimizing or maximizing a cutting effect of an electromagnetic energy emitting device, such as a laser. The fluid 34 of the present invention may be directed (e.g., as a spray, with or without pre-mixing with air, and/or as an atomized distribution of fluid particles) along with electromagnetic (e.g., laser) energy from an electromagnetic energy 32 toward a target surface 36, as shown in FIG. 4. Apparatuses including corresponding structure for directing fluid (e.g., an atomized distribution of fluid particles) and electromagnetic energy toward a target surface are disclosed in the above-referenced U.S. Pat. No. 5,741,247 (Att. Docket BI9001P) and in co-pending U.S. application Ser. No. 11/033,032, filed Jan. 10, 2005 and entitled ELECTROMAGNETIC ENERGY DISTRIBUTIONS FOR ELECTROMAGNETICALLY INDUCED DISRUPTIVE CUTTING (Att. Docket BI9842P), the entire contents of which are incorporated herein by reference. In certain instances, relatively large amounts of laser energy can be imparted into fluid particles sufficient to expand the fluid particles, wherein disruptive (e.g., mechanical and/or cutting) forces are applied to the target surface.

In view of the foregoing, it will be understood by those skilled in the art that the structures and methods of the present invention can facilitate operation of medical laser devices where fluid outputs are involved. The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention should not be limited by the disclosed embodiments, but is to be defined by reference to the appended claims.

What is claimed is:

1. A method of operating an electromagnetic energy emitting device, comprising:
providing a medical laser capable of receiving a fluid input and producing a fluid output;
receiving the fluid input;
controlling a temperature of the fluid;
producing the fluid output; and
combining the fluid output with electromagnetic energy output from the electromagnetic energy emitting device and causing the fluid to expand whereby disruptive forces are imparted onto the target.

2. The method as set forth in claim 1, wherein the electromagnetic energy emitting device comprises a medical laser.

3. The method as set forth in claim 1, wherein the controlling comprises:
receiving a target temperature;
sensing the temperature of the fluid output;
performing one of adding heat to the fluid and removing heat from the fluid according to the sensed temperature.

4. The method as set forth in claim 3, further comprising;
receiving a temperature delta;
providing a heat exchanger;
passing the fluid through the heat exchanger;
providing a heating component capable of causing the heat exchanger to increase the temperature of the fluid; and
causing the heating component to increase the temperature of the fluid when the sensed temperature is less than the target temperature minus the temperature delta.

5. The method as set forth in claim 4, further comprising;
providing a cooling component capable of causing the heat exchanger to decrease the temperature of the fluid; and
causing the cooling component to decrease the temperature of the fluid when the sensed temperature is greater than the target temperature plus the temperature delta.

6. The method as set forth in claim 1, wherein the controlling comprises:
providing a heat exchanger;
providing a heating component capable of causing the heat exchanger to increase the temperature of the fluid;
providing a cooling component capable of causing the heat exchanger to decrease the temperature of the fluid; and
selecting a low temperature;
selecting a high temperature;
passing the fluid through the heat exchanger;
causing the heating component to cause the heat exchanger to increase the temperature of the fluid when the sensed temperature of the fluid is less than the low temperature; and
causing the cooling component to cause the heat exchanger to decrease the temperature of the fluid when the sensed temperature of the fluid is greater than the high temperature.

7. The method as set forth in claim 1, further comprising sterilizing the fluid.

8. The method as set forth in claim 7, wherein the sterilizing comprises:
providing a lamp capable of radiating ultraviolet light; and
irradiating the fluid with the ultraviolet light.

9. The method as set forth in claim 1, wherein the receiving comprises filtering the fluid.

10. The method as set forth in claim 9, wherein the filtering comprises passing the fluid through a filter capable of removing particles larger than about 5 microns from the fluid.

11. An electromagnetic energy emitting device, comprising:
a receiving tube capable of receiving a fluid from a fluid input line;
a heat exchanger capable of performing one or more of adding heat to the fluid and removing heat from the fluid;
an output tube capable of receiving the fluid from the heat exchanger and of expelling the fluid in a direction of a target surface; and
a source of electromagnetic energy capable of directing electromagnetic energy to intersect with and expand at least a part of the expelled fluid whereby disruptive forces are imparted onto the target.

12. The method as set forth in claim 11, further comprising a sterilizing device capable of sterilizing the fluid, wherein:
the electromagnetic energy emitting device comprises a medical laser; and
the sterilizing device comprises an ultraviolet lamp.

13. The medical laser as set forth in claim 12, further comprising:
a heating component capable of causing the heat exchanger to add heat to the fluid, thereby increasing the temperature of the fluid;
a cooling component capable of causing the heat exchanger to remove heat from the fluid, thereby decreasing the temperature of the fluid;
a temperature sensor capable of measuring a temperature of the fluid; and
a controller capable of:
causing the heating component to effectuate an addition of heat to the fluid by the heat exchanger according to the measured temperature; and
causing the cooling component to effectuate a removal of heat from the fluid by the heat exchanger according to the measured temperature.

14. The medical laser as set forth in claim 13, wherein:
the controller is further capable of receiving a target temperature and a temperature delta;
the controller is capable of causing the heating component to effectuate an addition of heat to the fluid by the heat exchanger when the measured temperature is less than the target temperature minus the temperature delta; and
the controller is capable of causing the cooling component to effectuate a removal of heat from the fluid by the heat exchanger when the measured temperature is greater than the target temperature plus the temperature delta.

15. The medical laser as set forth in claim 13, wherein:
the heating component comprises a thermo-electric cooler; and
the cooling component comprises the thermo-electric cooler.

16. The medical laser as set forth in claim 12, wherein: the fluid input line comprises a filter capable of filtering the fluid; and the filter comprises pores not larger than about 5 microns in diameter.

17. A medical laser fluid temperature controller that controls temperature of a fluid, the medical laser fluid temperature controller comprising:
a processor capable of executing instruction sequences;
permanent memory capable of storing instructions;
a temperature sensor interface capable of receiving a measured fluid temperature value and of passing the measured fluid temperature value to the processor;
a temperature regulator capable of:
receiving a desired fluid temperature range; and
passing the desired fluid temperature range to the processor;
a thermo-electric control interface capable of controlling a thermo-electric control unit according to signals received from the processor;
a fluid temperature receiver instruction sequence stored in permanent memory that, when executed by the processor, minimally causes the processor to:
receive the measured fluid temperature value;
receive the desired fluid temperature range;
compare the measured fluid temperature value with the desired fluid temperature range to determine a comparison result; and
execute a thermo-electric control instruction sequence that passes signals to the thermoelectric control interface, thereby controlling the thermoelectric control device according to the comparison result to modify a temperature of the fluid; and
a fluid output coupled to route the modified-temperature fluid for combination with and expansion by a beam of electromagnetic energy on or above a target surface.

18. The medical laser fluid temperature controller as set forth in claim 17, wherein the desired temperature range comprises a high temperature setting and a low temperature setting.

19. The medical laser fluid temperature controller as set forth in claim 18, wherein the controlling of the thermo-electric control device comprises causing the thermo-electric control device to add heat to the fluid when the measured temperature is less than the low temperature setting.

20. The medical laser fluid temperature controller as set forth in claim 18, wherein the controlling of the thermo-electric control device further comprises causing the thermo-electric control device to remove heat from the fluid when the measured temperature is greater than the high temperature setting.
